(19) United States
(12) Patent Application Publication
(10) Pub. No.: US 2010/0152703 A1
(43) Pub. Date: Jun. 17, 2010

(54) System and method of efficacious body cavity washing

(76) Inventor: Hana R. Solomon, Columbia, MO (US)

Correspondence Address: Hana R. Solomon, MD, 1204 Sutton Dr., Columbia, MO, 65203, US

(21) Appl. No.: 12/316,620

(22) Filed: Dec. 15, 2008

(51) Int. Cl. A61M 3/02

(52) U.S. Cl. 604/514; 604/257

(57) ABSTRACT

Systems and methods of efficaciously washing body cavities, specifically including the nasal cavity wherein a fluid flow is governed in at least one of degree and direction of issuance. Governing of the degree of fluid flow can involve an applicator that is affixable to a fluid source, wherein the applicator can variably constrain the degree of fluid flow being issued. Governing of the direction of issuance can involve the applicator, when affixed to the fluid source, issuing the fluid flow in a specific direction, relative to the fluid source, and an indicator of the fluid source's general orientation relative to vertical.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

The present invention relates generally to systems and methods of washing a body cavity, and in particular to systems and methods of affecting a flow of fluid, including affecting the flow's disposition or degree, to provide efficacious washing of a body cavity.

2. Related Art

For many health or comfort care situations, utilizing an appropriate manner of applying a treatment can be nearly as critical to the treatment's effectiveness as correctly selecting a treatment that is suitable for the issue being treated. The consequences of misapplication can range from a relatively benign lack of relief, to a more serious initiation of an additional issue due to the misapplication, on up to an abandonment of treatment altogether, or even a switch to a differing, potentially even dangerous alternative treatment when the original treatment is still the best choice if applied properly. In many cases, the potential for misapplication is particularly prevalent when the treatment itself is a deceptively simple, everyday action. Washing of a body cavity is such an easily misunderstood treatment that the present invention provides systems and methods for ensuring its efficacious application.

A variety of body cavities, such as the nasal cavity or the vagina, are potentially benefitted by washing with externally applied techniques. A difficulty common among many of these cavities is that while the individual is, of course, quite familiar with the cavity in many senses, it is also generally not possible for the individual to see well within the cavity, nor to be exactly aware of the detailed internal structure and topography of their own cavity, since there are variations from person to person. Since many of the treatments available are preferably applied in particular dispositions relative to the cavity, and/or are best conducted in particular manners that are not easily regulated, especially when an individual is self treating, a significant potential for misapplication is common. Among the more frequent situations where body cavity washing can be beneficial, and a useful illustration of the difficulties addressed by the present invention, is the practice of washing (or cleansing) the nasal cavity. An understanding of the benefits of efficacious nasal washing has a long history in numerous cultures. Nasal washing or nasal irrigation is an ancient Ayurvedic technique known as Jala neti, which literally means “nasal cleansing” in Sanskrit (a classical language of South Asia). With origins based in the yoga tradition, nasal washing has been an accepted practice throughout India and South East Asia. Although not as historically common in Western cultures, these Eastern cultures have performed Jala neti as routinely as brushing one's teeth for centuries. Traditionally, a neti pot (which is a tea pot or Aladdin's lamp shaped vessel) has been used to flow salt water into one nostril, through the sinus cavities and out the other nostril. In the West, for over a century now, doctors have also come to appreciate the benefits of nasal washing. Alfred Laskiewicz, departmental Head of Pozna Otolaryngology Department (1932-1939) at the time, described conservative treatments of nasal irrigation from general hygiene to treatment of Scleroderma. The Proetz procedure has been used by ear, nose and throat surgeons for years to irrigate the sinuses after surgery.

Many forms of equipment have been utilized to effect nasal lavage including gravity flow vessels, pressure bottles, powered machines, misters, IV bags, sprays, squirts, flushers, bulbs, syringes, squeeze bottles, and even turkey basters. In addition to diversely configured physical modes of executing nasal washing, an assortment of differing types of wash solutions have also been employed. Most often, these solutions primarily consist of various salt solutions. Among those that have been studied are salt rinse formulations that are isotonic, hypertonic, or buffered, as well as those with additives such as colloidal silver, antibiotics and herbs. (An isotonic solution has the same salt concentration as the human body, a hypertonic solution has a greater salt concentration than the human body, and a buffered solution, in this context, is less acidic and is often generally better tolerated as well as potentially more effective.) The overall effectiveness of nasal washing has been repeatedly supported by the results of these scientific investigations. Currently, a hypertonic buffered saline solution has been shown to be most effective in treatment of congestion, along with removal of infectious and allergenic particles, but an isotonic buffered saline solution works well for overall daily washing, and many may still find significant beneficial effects even when using simple water if the more efficacious hypertonic or isotonic solutions are unavailable.

Over the past decade, a slowly growing expansion of interest in nasal cleansing has taken root in the United States. Among the factors contributing to this growth are the overall surge in appeal of preventative and alternative medicine, as well as a mounting trend towards an integrative approach to healthcare. Although antihistamines, decongestants, steroids, mucolytics, immune modulators, and antibiotics are already commonly prescribed for various nasal and sinus health issues, doctors have increasing also added the adjunct treatment of nasal irrigation for allergy suffers, post operative sinus surgery patients, and for patients with chronic sinusitis. Recently, proactive nasal washing is being recommended as a preventative approach for these health conditions. The benefits of nasal washing include refreshing, cleansing and moisturizing the nasal membranes. A lack of washing can produce substantial discomfort and health complications (such as allergic reactions, nasal congestion, sinus infections, post nasal drip, sore throat, asthma or nasal discharge) when not in optimal condition, as most people can personally attest. When washing with a hypertonic salt mixture, the nasal and sinus environment become more difficult for bacterial or viral growth. Presently, many products are available to enable implementing nasal washing more quickly, more easily, more effectively, and with less resulting mess. Even with such an extensive history of addressing the issue of nasal cleansing, substantial problems in ensuring that nasal cleansing is reliably efficacious remain unsolved, however. Among the principal problems remaining are that the vast majority of approaches do not account for the particulars of the physical arrangement of the nasal cavity, which impacts both the preferable manner and degree of nasal washing action. Most prior systems and methods enable introducing the wash solution to the nasal cavity without also enabling improved control and guidance of the manner of introduction (for example, washing pointing upwards can be uncomfortable), much less discernment, during the washing process itself, as to any aspect of whether the solution is being appropriately applied.

### SUMMARY OF THE INVENTION

At the core of much of the present invention's benefits is the recognition that the internal geography of the nasal cavity (as well as other body cavities) is not arranged to readily facilitate the washing of the cavity in general, much less specific washing objectives, from outside of the cavity. While it is possible to access the interiors of these cavities, one will still be essentially “driving in the dark” without a surgical or other procedure to provide inspection (obviously a problematic approach for an everyday action.) The exact particulars of the cavity's internal topography need to be properly addressed to achieve the desired results, and an understanding of both these challenges and the advantages provided by the present invention can be well illustrated by once again specifically considering the nasal cavity. In order to accomplish its various functions, the nasal cavity has evolved to pack a substantial amount of surface area into a relatively limited amount of volume. Not only is the nasal cavity interior volume demarcated by a complex manifold, but the conduits between the nasal cavity and the other spaces it interconnects with (e.g. sinuses and the inner ear) may provide only diminutive ostia for fluids to communicate between. An important objective of nasal washing is to improve drainage from these spaces, through the nasal cavity, and provide relief from excess pressure. The present invention facilitates and enhances the intercommunication of fluids and pressures between the nasal cavity and the other spaces by providing modes of administering a flow of fluids that influence the ostia to open. Even if practical, directly applying a washing fluid flow to the ostia, via the nasal cavity, is generally not advantageous. The ostia's minute dimensions, along with their and the nasal cavity's tendency to already contain blocking, or potentially blocking debris, produces a likelihood that they become even more obstructed because the directly applied fluid flow can pack the debris into the ostia. Furthermore, the desired normal mucous flow direction is out through the ostia into the nasal cavity, not back into the sinuses or inner ear, as would occur when a fluid flow is directly applied to the ostia from within the nasal cavity. Among the key achievements of the present invention is its ability to govern the conduct of a fluid flow administered to the nasal cavity so that the ostia are influenced to open and provide intercommunication between the nasal cavity and the other intra-head spaces.

The present invention, by governing a fluid flow's specific conduct into and at least partially through the nasal cavity, utilizes Bernoulli's principle to enact its impact on the ostia. Bernoulli's principle, roughly synopsized, states that as a fluid flow's speed increases its surface pressure decreases. Hence, when a flow of fluid is conducted into and at least partially through the nasal cavity with a suitable trajectory and velocity so that the ostia encounter the reduced surface pressure due to Bernoulli's principle, passage of fluid and other matter within the intra-head passages that interconnect with the nasal cavity will be influenced to drain into the nasal cavity via the ostia. It can be decisive that appropriate conduct of the fluid flow be readily achieved by the user, or the desired effect upon the ostia will not be reliably realized. The appropriate conduct involves directing the flow of fluid across the floor of the nasal cavity so it can flow past the sinus and other ostia, creating pressure differentials that encourages the draining of the sinus' contents. Incorrectly directing the fluid flow upward, rather than backward, may aim it directly at the openings to the sinuses, potentially not only blocking outward flow through the ostia, but possibly also causing transient discomfort as pressure is forced into inadequate spaces. The avoidance of this prospect of discomfort is another primary benefit of the present invention.

A significant attribute of embodiments of the present invention are their provision of at least one flow influencing feature that produces effects upon a fluid flow administered to the user to achieve the benefits described. Various embodiments of the present invention govern the conduct of the fluid flow by, among other manners, indicating the orientation of the fluid flow relative to vertical with a first flow influencing feature and by providing a variable restrictability of the degree of the fluid flow with a second flow influencing feature. By receiving an indication of the fluid flow's orientation relative to vertical, the user is able to properly direct the flow in the manner required while still being able to hold their head and neck in a natural upright and neutral position, thereby ensuring both that the user is comfortable (to promote regular use) and that the user is able to readily replicate the proper positioning of the flow relative to their head by allowing them to administer the flow with their head held in an repeatedly attainable, easily maintained neutral position. Among other ways, the present invention provides manners of suitably directing the fluid flow, relative to vertical, by providing the user an indication of the orientation, relative to vertical, of a source of the fluid flow being administered. The source is interrelated with an applicator that governs the issuing fluid flow by at least one of influencing the direction of the issued fluid flow and variably restricting the fluid flow's degree. In regard to influencing the direction of the fluid flow, some embodiments of the applicator can govern the fluid flow to issue in a known, or even specified, direction relative to the source; which, in combination with the indication of the source's orientation relative to vertical, allows the user to attain and maintain the fluid flow in a given orientation relative to vertical. Hence, by holding the head neutral, arranging the fluid flow in a selected orientation (generally perpendicular) relative to vertical, and suitably placing the applicator and source at the nasal cavity opening the user can reliably and easily direct the fluid flow correctly for efficacious nasal washing.

Various embodiments of the present invention include, sometimes as an optional feature, a second flow influencing feature for governing the conduct of the fluid flow: an applicator able to variably restrict the cross-section of a passage that the fluid flow traverses in order to impact its degree of flow. Some embodiments of the present invention, such as those that include a resiliently deformable reservoir, are capable of providing a fluid flow that can be highly changeable in degree. While this changeability can provide a capability that is advantageous to some, who appreciate the option to flexibly choose how great a degree of flow they will to attempt to apply, others will prefer an ability to ensure that the flow is at least somewhat constrained so that it is, for example, more predictable in degree or requiring less concentration or less user ability to exert physical control of the reservoir deformation. In certain circumstances, the fluid flow degree's restrictability can be critical for a given user to be able to benefit from a nasal washing treatment, such as when a medical condition can present negative complications if the body cavity washing is too vigorous, e.g. following nasal surgery. A major factor in the long-term success of body cavity washing can be conditional upon the user utilizing the washing treatment on a consistent basis, so that any manner of washing that presents a significantly improved comfort level can, by promoting such consistent use, result in a substantially more efficacious treatment. The present invention can accomplish such a substantially improved treatment result by its provision of features that can also greatly improve the comfort level of the user.

Other objects and features will be in part apparent and in part pointed out hereinafter.

### BRIEF DESCRIPTION OF THE DRAWINGS

FIG. 1 depicts a schematic cross-section view of an orientation indicating embodiment of the present invention disposed relative to a user in a specific orientation relative to vertical and includes a schematic cross-section view of the user's nasal cavity.

FIG. 2 depicts an expanded schematic cross-section view of a user's nasal cavity with relevant anatomical aspects detailed.

FIG. 3 depicts a schematic exploded view of components of an embodiment of an applicator according to the present invention

FIG. 4 depicts a schematic cross-section view of an assembled embodiment of an applicator according to the present invention.

### DETAILED DESCRIPTION OF THE PREFERRED EMBODIMENTS

In the following description, identical numbers indicate identical elements. Where an element has been described in one Figure, and is unaltered in detail or relation in any other Figure, said element description applies to all Figures.

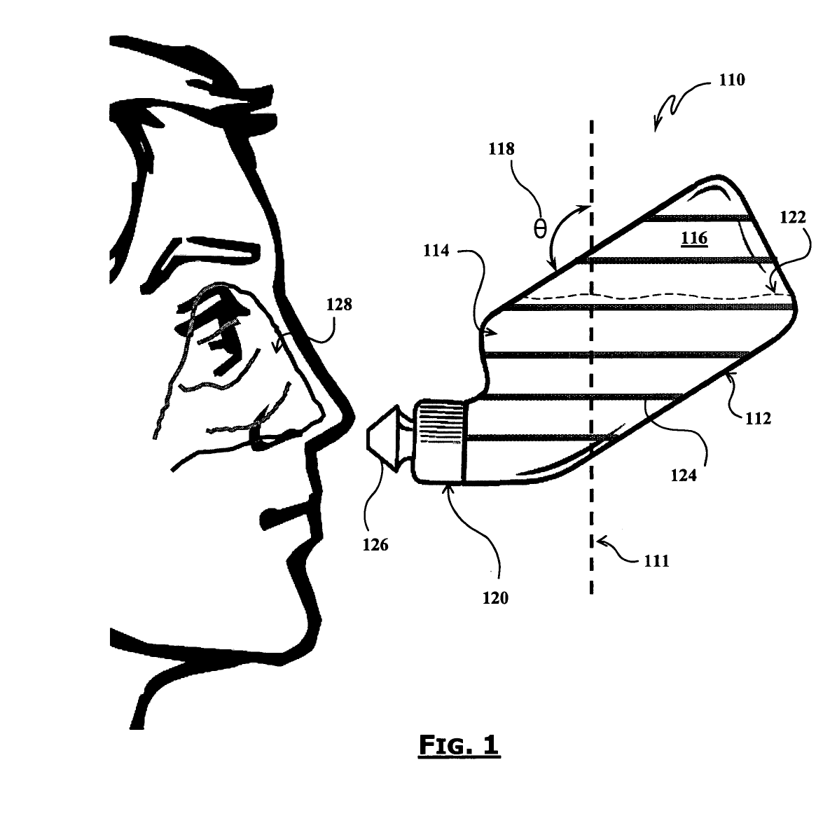

A first orientation indicating embodiment 110 of the present invention, shown in FIG. 1, is depicted in a representative usage situation, about to be repositioned towards the nostril of the individual user depicted. The reference attribute, relative to vertical (indicated by the dashed line 111), for the first orientation indicating embodiment 110 is predicated to be the longitudinal wall 112 of the fluid source's main body 114. In certain embodiments the fluid source's main body 114 is also a reservoir for those embodiments. For the first orientation indicating embodiment 110 depicted in FIG. 1, the main body 114 is also serving to provide a reservoir 116. The longitudinal walls 112 (which may comprise one or more interconnected sections) define the longest dimension of the main body 114 and hence are a straightforward selection as defining the orientation of the first orientation indicating embodiment 110. Hence, when the longitudinal wall 112 is shown as disposed at an angle θ 118, relative to vertical, the first orientation indicating embodiment 110 will then be described as being oriented at an angle θ 118, relative to vertical, as well. A fluid level in the reservoir 116 is indicated by the surface 122 of the fluid contained within the reservoir 116. At least some portions of the longitudinal walls 112 are constructed to be at least partially transparent so that the user can see the fluid surface 122. Since the fluid surface 122, in response to gravity, will assume a general disposition perpendicular to vertical 111, comparison of the orientation of the first orientation indicating embodiment 110 to the orientation of the fluid surface 122 will reveal the orientation of the first orientation indicating embodiment 110 relative to vertical 111. For appropriate use of the first orientation indicating embodiment 110, it is not necessary to ascertain its orientation in general, but merely to ascertain if it is in the desired orientation, relative to vertical 111, in order to properly direct the fluid flow. At least one, and usually a plurality of, aligned indicia 124 are arranged on at least a portion of the periphery of the main body 114, particularly including at least some of the at least partially transparent portions of the longitudinal walls 112. By visually aligning, at least roughly, at least one indicia with the fluid surface level 122, the user can align and maintain the first orientation indicating embodiment 110 in a desired orientation relative to vertical. Disposed in such a desired orientation relative to vertical, the first orientation indicating embodiment 110 is located with the applicator 126 in contact with the nostril so that the fluid flow can be administered in the desired direction to a nasal cavity 128.

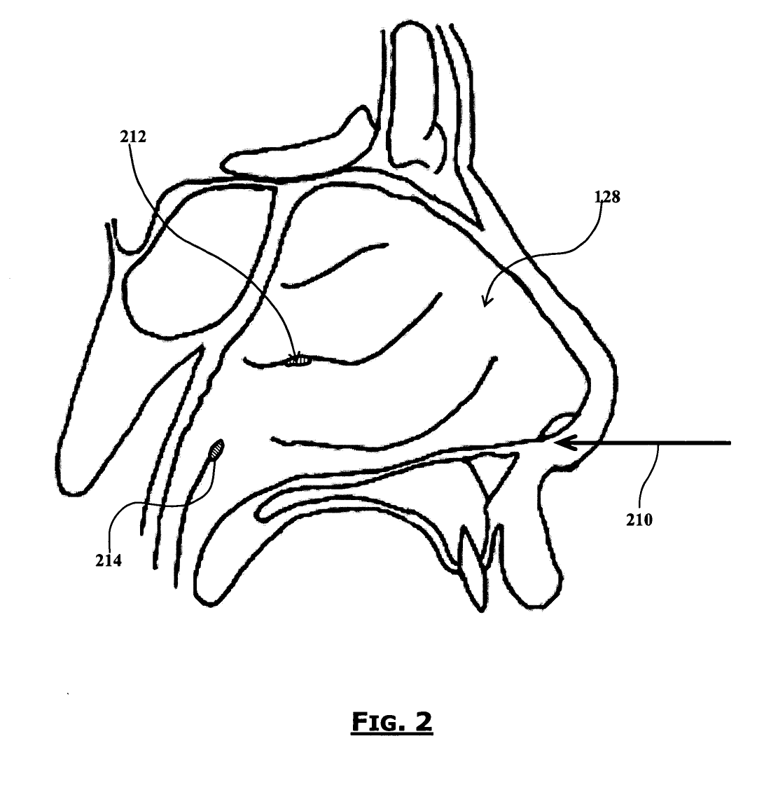

The nasal cavity 128 is depicted in substantially greater detail in the cross-section shown in FIG. 2. The desired direction 210 and disposition of fluid flow administration is shown as entering along the floor of the nasal cavity 128, where it will interact with the complicated internal space within the nasal cavity 128. The majority of the details of this complicated internal space will not be explicated in depth herein, since it is well known to those of skill in the art and the details are easily available by consultation of an innumerable assortment of references. However, two specific aspects of this internal space will be described in detail since they are particularly germane to the benefits provided by the present invention. As described earlier, the ostia within the nasal cavity 128 are diminutive orifices that enable intercommunication between the nasal cavity 128 and other spaces within the head, and their specific locations impact the difficulties encountered in clearing these other spaces via the nasal cavity 128 and in being able to promote their opening when blocked. A sinus ostia 212 and the ostia interconnecting the inner ear 214 are shown in their general dispositions in the schematic drawing depicted in FIG. 2, although it should be understood that these locations can and often will vary, at least somewhat, from individual to individual, with their basic dispositions usually corresponding at least approximately with the FIG. 2 depiction. It is clear that direct access to these ostia is not readily available even to a trained medical professional, much less to an everyday, self-treating layman.

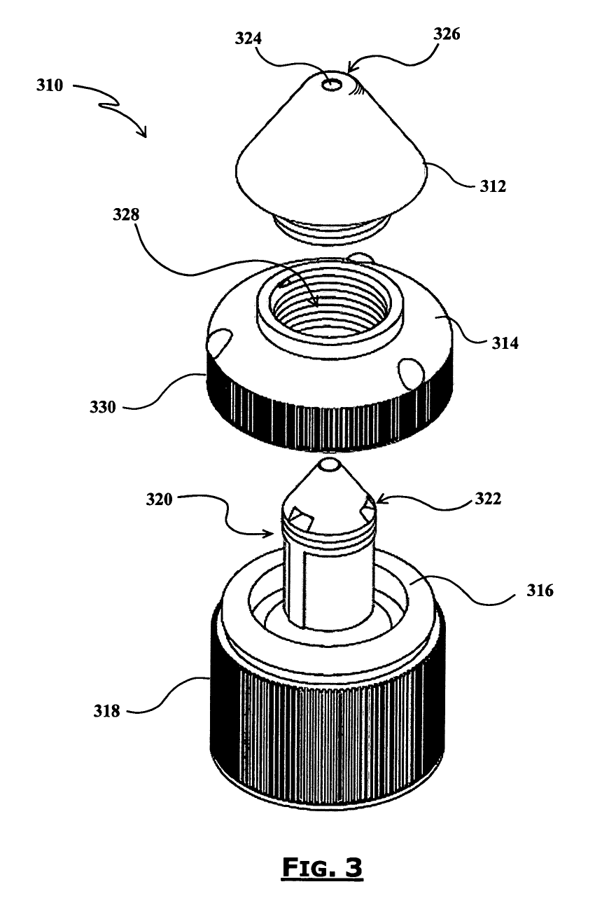

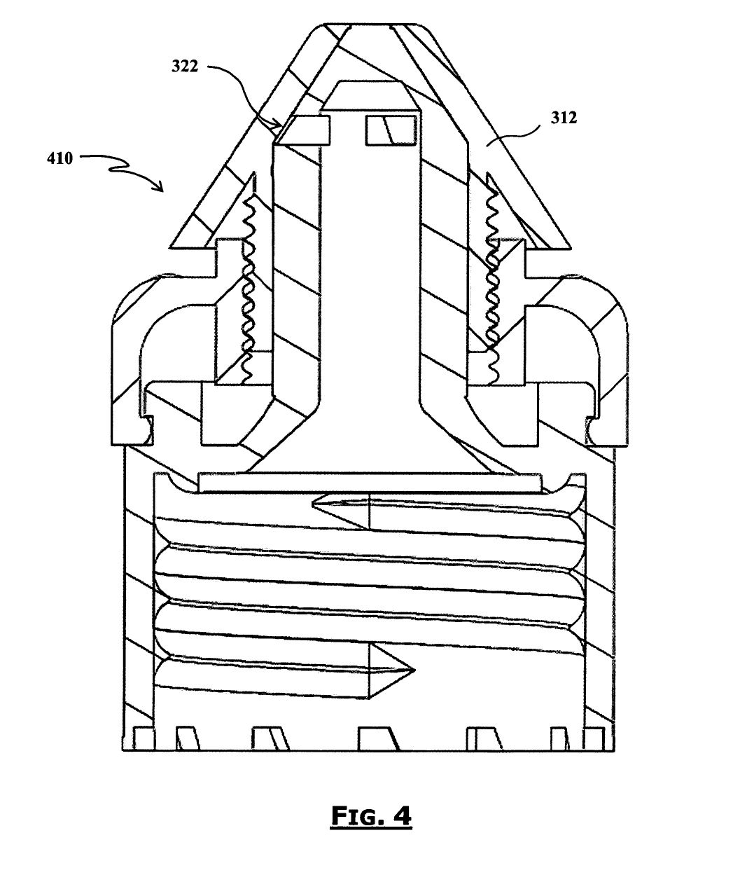

As discussed earlier, an efficaciously conducted fluid flow according to the present invention can include a manner of governing the degree of fluid flow, such as by utilization of the first variably restrictable applicator 310, depicted in an exploded view in FIG. 3. the first variably restrictable applicator 310 comprises three primary sub-parts, an applicator tip 312, and intermediate variable restrictor 314, and an applicator base 316. The applicator base 316 interconnects with a throat portion 318 to the main body 114, and includes an internal passage (not shown) that, when interconnected with the main body 114, allows fluid within the reservoir 116 to access a conduit 320 which terminates in at least one port 322. When the first variably restrictable applicator 310 is assembled, as shown in cross-section in FIG. 4, the at least one port 322 opens into an interior volume (not shown) of the applicator tip 312. The interior volume of the applicator tip 312 interconnects with an aperture 324 through which the fluid flow can emerge. An exterior terminus 326 of the applicator tip 312 is shaped to allow a close fit with the nasal cavity orifice, without allowing substantial, potentially damaging entry to the nasal cavity 128. The intermediate variable restrictor 314 both fits around and upon the applicator base 316, often though not exclusively with a snap and ridge interconnection, and provides an interior channel 328 through which the conduit 320 is inserted until the at least one port 322 enters the interior volume of the applicator tip 312. The applicator tip 312 is interconnected with the intermediate variable restrictor 314, often though not exclusively with a screw-thread interconnection, so that rotational movement of the intermediate variable restrictor 314, for example enacted by gripping and torquing the ridges 330, will move the applicator tip 312. When the intermediate variable restrictor 314 is turned relative to the applicator base 316, the applicator tip 312 is movable both closer and farther, depending on the direction of turning, form the applicator base 316, which in turn provides progressively lesser or greater, respectively, space between the at least one port 322 and the interior surface of the applicator tip 312. When more space is provided, a greater degree of fluid flow is permitted, and when lesser space is provided, fluid flow is thereby restricted to being only a lesser degree.

An assembled applicator cross-section view 410 of the first variably restrictable applicator 310 in FIG. 4 depicts the applicator tip 312 in a maximally fluid flow restricting disposition. In this maximally fluid flow restricting disposition, it can be seen that the at least one port 322 is almost flush with the interior surface of the applicator tip 312, and hence only a minimal amount of fluid flow is able to pass through the at least one port 322. Although not shown, it is readily understandable that by moving the applicator tip 312 further away from the at least one port 322, as described immediately above, progressively greater amounts of fluid flow are able to pass.

In view of the above, it will be seen that the various objects and features of the invention are achieved and other advantageous results obtained. The examples contained herein are merely illustrative and are not intended in a limiting sense.

What is claimed is:

1. A system of efficacious body cavity washing comprising:
an applicator interrelated with a source of fluid suitable for washing a body cavity, said fluid source and interrelated applicator capable of issuing a flow of the fluid that is at least partially governable in at least one of direction and degree by said applicator;
said applicator being fixable, relative to the fluid source, in a first applicator disposition that governs the fluid flow to generally issue in a first direction relative to the fluid source;
said fluid source and applicator further including at least one of first and second flow influencing features, respectively, wherein said first flow influencing feature is a fluid source spatial orientation indicator and said second flow influencing feature is a variable restrictor of the fluid flow's degree.

2. The system of efficacious body cavity washing according to claim 1 wherein said first flow influencing feature is requisite and said second flow influencing feature is optional.

3. The system of efficacious body cavity washing according to claim 1 wherein said second flow influencing feature is requisite and said first flow influencing feature is optional.

4. The system of efficacious body cavity washing according to claim 1 wherein said first flow influencing feature's spatial orientation indicator can indicate the fluid source's general orientation relative to vertical.

5. The system of efficacious body cavity washing according to claim 4 wherein said fluid source comprises a fluid reservoir with a reservoir housing, at least a portion of said reservoir housing revealing fluid within the reservoir and including level indicia arranged so that aligning the reservoir fluid's surface with the level indicia aligns the reservoir housing in a first orientation relative to vertical, such that the fluid flow issues in a second direction relative to vertical.

6. The system of efficacious body cavity washing according to claim 5 wherein said second direction is perpendicular to vertical.

7. The system of efficacious body cavity washing according to claim 6 wherein the reservoir housing's spatial disposition is largely delineated by a primary housing axis generally disposed at an angle of about 120 degrees relative to said fluid flow first direction.

8. The system of efficacious body cavity washing according to claim 7 wherein said level indicia are generally disposed at an angle of about 120 degrees relative to said first housing axis.

9. The system of efficacious body cavity washing according to claim 1 wherein said second flow influencing feature's variable restrictor involves a selectively constrainable conduit which said issuing fluid flow travels through.

10. The system of efficacious body cavity washing according to claim 9 wherein the conduit is selectively constrainable by selective relative rotation of first and second components of said applicator, and, optionally, said selective constrainability involves at least one of continuous and discrete variations in constraint actuated by continuous and discrete, respectively, relative rotations of the first and second applicator components.

11. A method of efficaciously washing a body cavity comprising the steps of:
interrelating an applicator with a source of fluid suitable for washing a body cavity;
issuing a flow of said fluid from the source and at least partially governing at least one of a direction and degree of the flow with said applicator;
fixing the applicator in a first applicator disposition, relative to the fluid source, that governs the fluid flow to generally issue in a first direction relative to the orientation of the fluid source; and
utilizing at least one of first and second flow influencing features of the fluid source and applicator, respectively, wherein said first flow influencing feature can indicate at least one aspect of the fluid source's spatial orientation and said second flow influencing feature can variably restrict the fluid flow's degree.

12. The method of efficaciously washing a body cavity according to claim 11, wherein utilizing said first flow influencing feature is requisite and utilizing said second flow influencing feature is optional.

13. The method of efficaciously washing a body cavity according to claim 11, wherein utilizing said second flow influencing feature is requisite and utilizing said first flow influencing feature is optional.

14. The method of efficaciously washing a body cavity according to claim 11, wherein said first flow influencing feature can indicate the fluid source's general spatial orientation relative to vertical.

15. The method of efficaciously washing a body cavity according to claim 14, wherein said fluid source comprises a fluid reservoir with a reservoir housing, at least a portion of said reservoir housing revealing fluid within the reservoir and including level indicia arranged so that aligning the reservoir fluid's surface with the level indicia aligns the reservoir housing in a first orientation relative to vertical, such that the fluid flow issues in a second direction relative to vertical.

16. The method of efficaciously washing a body cavity according to claim 15, wherein said second direction is perpendicular to vertical.

17. The method of efficaciously washing a body cavity according to claim 16, wherein the reservoir housing's spatial disposition is largely delineated by a primary housing axis generally disposed at an angle of about 120 degrees relative to said fluid flow first direction.

18. The method of efficaciously washing a body cavity according to claim 17, wherein said level indicia are generally disposed at an angle characterizable as either of 120 and 60 degrees relative to said first housing axis.

19. The method of efficaciously washing a body cavity according to claim 11, wherein said second flow influencing feature's variable restrictor involves a selectively constrainable conduit through which the fluid flow passes.

20. The method of efficaciously washing a body cavity according to claim 19, wherein said conduit is constrainable by relative rotation of first and second applicator components, and optionally, said constraining is at least one of continuously and discretely variable via continuous and discrete, respectively, relative rotations of the first and second applicator components.

21. The method of efficaciously washing a body cavity according to claim 11, further comprising the step of providing at least one of:
a) a system capable of effecting the present method,
b) guidance in executing the present method,
c) guidance how to provide a system capable of effecting the present method,
d) and combinations thereof.

22. A system adapted for efficaciously washing a nasal cavity with a suitable fluid comprising:
an applicator affixable to a fluid reservoir such that the general conduct of a flow of fluid issuable by the reservoir is at least partially governable by the applicator, said governing including guiding the fluid flow to generally emanate from the applicator in a specific direction, said applicator including at least one exterior aspect adapted for administering said fluid flow to a nasal cavity;
said reservoir comprising a first flow influencing feature able, when the applicator is affixed to the reservoir, to indicate the specific direction's general orientation relative to vertical by indicating the reservoir's general orientation relative to vertical.

23. A system adapted for efficaciously washing a nasal cavity with a suitable fluid according to claim 22, wherein said applicator governability further includes variably constraining a degree of said fluid flow.

24. A system adapted for efficaciously washing a nasal cavity with a suitable fluid according to claim 23, wherein said variable constraining utilizes a selectively constrictable fluid flow conduit.

25. A system adapted for efficaciously washing a nasal cavity with a suitable fluid according to claim 22, wherein said fluid flow conduit is constrictable by selective relative rotation of first and second components of said applicator, and, optionally, said selective constrainability involving at least one of continuous and discrete variations in conduit constriction actuated by continuous and discrete, respectively, relative rotations of the first and second applicator components.

26. A method of efficaciously washing a nasal cavity with a suitable fluid comprising the steps of:
affixing an applicator to a fluid reservoir such that the general conduct of a flow of fluid issuable by the reservoir is at least partially governable by the applicator, said applicator including at least one exterior aspect adapted for administering said fluid flow to a nasal cavity;
guiding the flow of fluid, with said applicator's at least partial governing, to emanate from the applicator in a specified direction; and
facilitating disposing said specified direction in a selected general orientation relative to vertical by indicating the reservoir's general orientation relative to vertical.

27. A method of efficaciously washing a nasal cavity with a suitable fluid according to claim 26, wherein said applicator governability further includes variably constraining a degree of said fluid flow.

28. A method of efficaciously washing a nasal cavity with a suitable fluid according to claim 27, wherein said variable constraining utilizes a selectively constrictable fluid flow conduit.

29. A method of efficaciously washing a nasal cavity with a suitable fluid according to claim 26, wherein said fluid flow conduit is constrictable by selective relative rotation of first and second components of said applicator, and, optionally, said selective constrainability involving at least one of continuous and discrete variations in conduit constriction actuated by continuous and discrete, respectively, relative rotations of the first and second applicator components.

30. A system adapted for efficaciously washing a nasal cavity with a suitable fluid comprising:
a fluid reservoir and an affixable adaptor that includes at least one exterior aspect adapted for administering fluids to a nasal cavity, said affixing enabling the adaptor to at least partially govern general conduct of a flow of fluid issuable by the reservoir, said governing optionally including guiding the fluid flow to generally emanate from the applicator in a specific direction;
said reservoir comprising a resiliently deformable housing able to issue a fairly unfettered degree of fluid flow when said housing is deformed; and
said applicator including a second flow influencing feature able to variably constrain the fluid flow's degree.

31. A system adapted for efficaciously washing a nasal cavity with a suitable fluid according to claim 30 when the affixed applicator's governing is guiding the fluid flow to generally emanate in said specific direction, further comprising a first flow influencing feature that can indicate a spatial orientation, relative to vertical, of the emanated fluid flow by indicating the spatial orientation, relative to vertical, of the fluid reservoir.

32. A system adapted for efficaciously washing a nasal cavity with a suitable fluid according to claim 31 further comprising a reservoir housing that includes at least a portion that reveals fluid within the reservoir and includes specifically arranged fluid level indicia, wherein a specific spatial orientation relative to vertical of the fluid reservoir is effectible by aligning a surface of the fluid within the reservoir with at least one of the fluid level indicia.

33. A system adapted for efficaciously washing a nasal cavity with a suitable fluid according to claim 32, wherein a spatial disposition of the reservoir housing is largely delineated by a housing primary axis, and said level indicia are generally disposed at an angle of about 120 degrees relative to said primary axis.

34. A method of efficaciously washing a nasal cavity with a suitable fluid comprising the steps of:
affixing an adaptor that includes at least one exterior aspect adapted for administering fluids to a nasal cavity to a fluid reservoir, said affixing enabling the adaptor to at least partially govern general conduct of a flow of fluid issuable by the reservoir, said governing including guiding the fluid flow to generally emanate from the applicator in a specific direction;
issuing an unregulated degree of fluid flow by deforming a resilient reservoir housing; and
variably constraining the fluid flow's degree with a second flow influencing feature of the applicator.

35. A method of efficaciously washing a nasal cavity with a suitable fluid according to claim 34, further comprising the step of utilizing a first flow influencing feature for additionally guiding the fluid flow's specific direction of emanating to realize a first specified orientation, relative to vertical, by aligning the fluid reservoir in a second specified orientation, relative to vertical.

36. A method of efficaciously washing a nasal cavity with a suitable fluid according to claim 35, wherein said first flow influencing feature involves the fluid reservoir further comprising a reservoir housing that includes at least a portion that reveals fluid within the reservoir and includes specifically arranged fluid level indicia such that a specific spatial orientation relative to vertical of the fluid reservoir is effectible by aligning a surface of the fluid within the reservoir with at least one of the fluid level indicia, and further comprising the step of disposing the emanating fluid flow in a specific orientation, relative to vertical, by aligning the fluid surface within the reservoir with at least one of the fluid level indicia.

37. A method of efficaciously washing a nasal cavity with a suitable fluid according to claim 36, wherein a spatial disposition of the reservoir housing is largely delineated by a housing primary axis, and said level indicia are generally disposed at an angle of about 120 degrees relative to said primary axis.

38. A nasal irrigator comprising:
a reservoir having a generally resiliently deformable housing for retaining irrigating fluid and an applicator affixable to the reservoir, a flow of said irrigating fluid being issuable by the reservoir when the housing is deformed, said flow being at least partially governable by the applicator when the applicator is affixed to the reservoir, said governing including both variably constraining the degree of the fluid flow and guiding the fluid flow to generally emanate from the applicator in a specific direction relative to the reservoir, said applicator including at least one exterior aspect adapted for administering the fluid flow to a nasal cavity, said housing including an indicator of the housing's orientation relative to vertical;
said housing orientation indicator comprising an at least partially transparent portion of the reservoir housing revealing fluid within the reservoir and including fluid level indicia specifically arranged so that aligning a surface of the fluid within the reservoir with at least one of the fluid level indicia disposes the fluid reservoir in a specific spatial orientation relative to vertical; and
said fluid flow degree variable constraining utilizes a fluid flow conduit that is selectively constrictable by selective relative rotation of first and second applicator components, and, optionally, said selective constraining involving at least one of continuous and discrete variations in conduit constriction actuated by continuous and discrete, respectively, relative rotations of the first and second applicator components.
